(19) United States
(12) Patent Application Publication
(10) Pub. No.: US 2012/0225471 A1
(43) Pub. Date: Sep. 6, 2012

(54) Method for producing a composite material

(76) Inventors: Gavin McIntyre, Troy, NY (US); Allison Poetzsch, Ravena, NY (US); Sue Van Hook, Cambridge, NY (US); Daniel Flagg, Schenectady, NY (US)

(21) Appl. No.: 12/932,640

(22) Filed: Mar. 2, 2011

(51) Int. Cl. C12N 1/14

(52) U.S. Cl. 435/254.1

(57) ABSTRACT

A plant essential oil emulsion is used to disinfect the raw material used in making a self-supporting composite material formed by the growth of the hyphae of a fungus into and about the discrete particles of the raw material.

This invention relates to a method for producing a composite material. More particularly, this invention relates to a method for producing a sterilized composite material using fungal tissue. Still more particularly, this invention relates to a method of disinfecting bulk materials that can later be inoculated with a filamentous fungus for composite materials production or cultivation of food (mushrooms).

Published US Patent Application US 2008/0145577 describes a method of making a composite material comprising the steps of forming an inoculum including a preselected fungus (mycelium culture); forming a mixture of a substrate of discrete particles and a nutrient material capable of being digested by the fungus; adding the inoculum to the mixture; and allowing the fungus to digest the nutrient material in the mixture over a period sufficient to grow hyphae and to allow the hyphae to form a network of interconnected mycelia cells through and around the discrete particles thereby bonding the discrete particles together to form a self-supporting composite material.

The discrete particles that may be employed include vermiculite and perlite where the composite material is to be used as a fire-resistant wall; straw, hay, hemp, wool, cotton, rice hulls and recycled sawdust where the composite material is to be used for insulation and strength is not a necessary criteria; and synthetic insulating particles, such as, foam based products and polymers. Other discrete particles that may be used as a bulking agent include seed hulls, such as of buckwheat, oat and cotton, as well as fiberized material, such as cotton and sorghum.

One known composite product that has been on the market is comprised predominantly of low-value agricultural byproducts that are bound using the vegetative growth of a filamentous fungus (mycelium). Such products have been marketed under the trademark ECOCRADLE for use as a biodegradable non-paper packaging and shipping buffers for physical and thermal protection of items during shipping.

Where the discrete particles are obtained from plant and/or botanical vegetable waste, commercial embodiments of the known method of making the composite materials employ a pasteurization step to clean the raw materials prior to inoculation with the mycelium culture for use in the growing steps. However, such pasteurization steps can be expensive, time consuming and effective for only 5 to 7 days.

Accordingly, it is an object of the invention to provide a disinfection process for the raw materials used in making producing a composite material using fungal tissue.

It is another object of the invention to reduce the costs of manufacturing composite materials using fungal tissue.

It is another object of the invention to reduce the time required to produce a sterilized composite material using fungal tissue.

Briefly, the invention provides a method for producing a composite material wherein a substrate of a raw material composed of discrete particles of an agricultural byproduct is provided with a plant essential oil emulsion, such as a phenol and/or aldehyde emulsion, in an amount and for a time sufficient to effectively inhibit microbial growth on the substrate. Thereafter, an inoculum that includes a preselected fungus is added to the mixture of raw material and phenol and/or aldehyde emulsion and the inoculated fungus is allowed to grow hyphae. During growth, the hyphae form a network of interconnected mycelia cells through and around the discrete particles thereby bonding the discrete particles together to form a self-supporting composite material.

In another embodiment, the phenol and/or aldehyde emulsion may be removed from the mixture of raw material and emulsion prior to inoculation with the fungus, for example, so that the emulsion or remainder of the emulsion may be reused to disinfect a fresh batch of raw material.

In still another embodiment, the phenol and/or aldehyde emulsion may be removed from the mixture of raw material and emulsion and recycled into the mixture prior to inoculation with the fungus.

Further, upon completion of the disinfection stage, the substrate may be delivered to a devolitization station in order to de-volatize the remaining phenols and aldehydes as well as any water, calcium oxide (lime), or vegetable oil contained in the substrate. Thereafter, the substrate is delivered to the inoculation station.

The method is particularly suited to the use of ligninocellulosic materials and cellulosic waste, such as cotton gin waste, as the raw material. However, other low-value agricultural byproducts may be used. It has been found that the substrate that can be disinfected with the plant essential oil emulsion should be comprised of the following, examples of the constituents are found in parentheses. A mixture of heterogeneous and/or homogenous particles, nutritional (lignocellulosic material) or non-nutritional (synthetic polymer), that is supplemented with an additional carbon (maltodextrin, starch), and/or nitrogen (peptone, nutritional yeast) and/or mineral source (CaCl₂, MgSO₄) such that the substrate can maintain the growth of a selected filamentous fungus or microbe.

The emulsion that is used in the method of the invention contains plant based phenols (such as thymol and carvicrol), aldehydes (such as, cinnamdehyde), or a combination thereof. In particular, the emulsion includes a plant essential oil (PEO) selected from the group consisting of cinnamon, clove, lemongrass, oregano, savory, spearmint, tea tree, and thyme and is provided in volumetric concentrations of 0.1 to 2.0% in water. Generally, the minimal inhibitory concentration can be selected for the organism, for example, bacteria is not an issue while mold is, and mold can be inactivated with 0.03% cinnamon oil or 0.1% thyme oil.

The fungus is either preselected based on enzymatic capabilities, as such, the substrate does not have to be post processed prior to inoculation (tissue introduction), or is genetically modified and then used to inoculate the disinfected substrate.

The inoculum includes a fungus such as a fungus selected from the white rot decomposers that produce the desired enzyme. The families are Polyporaceae and Ganodermataceae, while the order is Aphyllophorales.

For example, the fungus is a white rot basidiomycetes selected from the group consisting of the two species, Ganoderma resinaceum and Fomes fomentarius which are known as white rot decomposers and metabolize the aromatic biopolymer lignin and the two species, Ganoderma lucidum and Ganoderma brownii which are known for producing peroxidases and/or dehydrogenases for metabolizing phenols and aldehydes, respectively.

These and other objects and advantages of the invention will become more apparent from the following detailed description taken in conjunction with the accompanying drawings wherein:

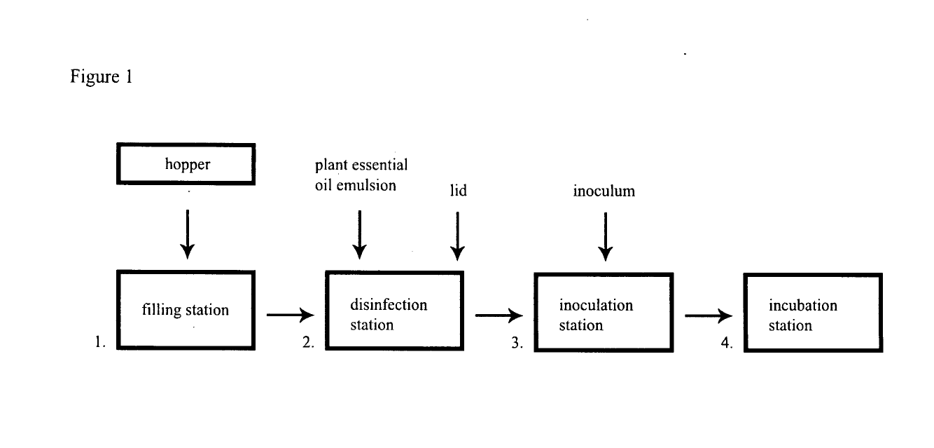

FIG. 1 illustrates a schematic of a process in accordance with the invention;

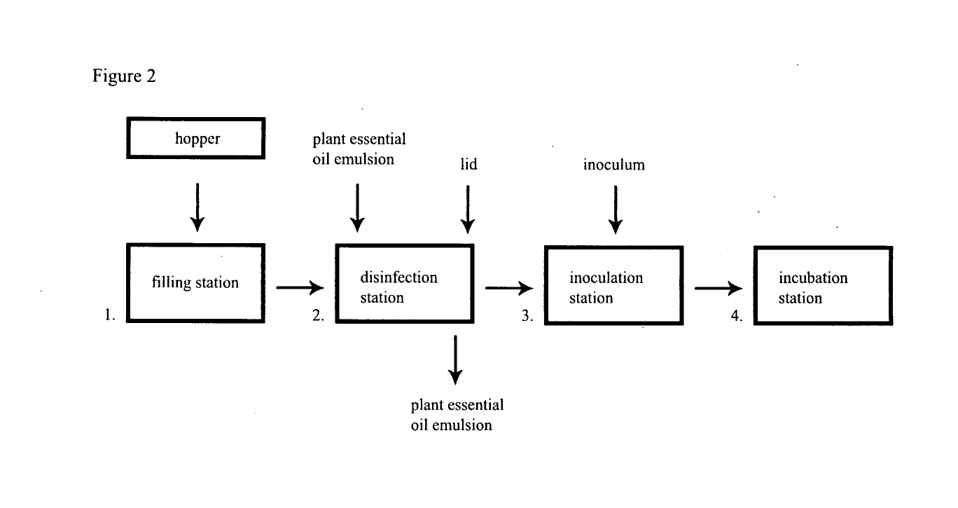

FIG. 2 illustrates a schematic of a process in accordance with the invention in which the plant essential oil is removed prior to the step of inoculation; and

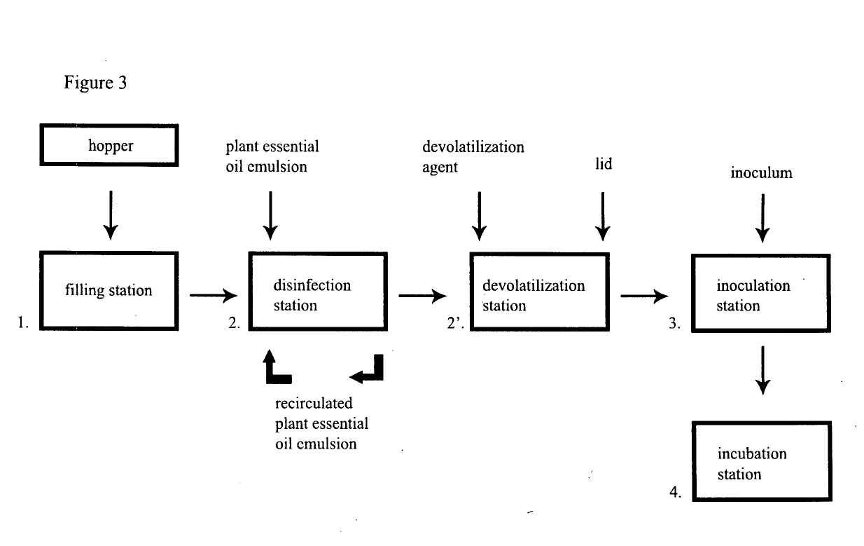

FIG. 3 illustrates a schematic of a process in accordance with the invention in which the substrate is subjected to a devolitization step to remove remaining phenols and aldehydes.

Referring to FIG. 1, the method for producing a composite material employs a filling station 1 (or stage), a disinfection station 2 (or stage), an inoculation station 3 (or stage) and an incubation station 4 (or stage). The method may be performed in a batch-wise manner of a continuous manner or a combination of each manner.

The method includes an initial step of delivering a substrate in the form of raw material composed of discrete particles of an agricultural byproduct, such as cotton gin waste, from any suitable source to the filling station 1 in which the raw material may be delivered in a batch manner via a hopper into an open top tray (not shown) that serves as a form for the final product to be produced in accordance with the methods described in U.S. Ser. No. 12/001,556, filed Dec. 12, 2008.

The filling station 1 also includes a conveyer of suitable construction under the hopper for conveying the raw material filled tray away from the filling station 1.

A series of trays may be filled in a sequential manner and conveyed in spaced apart relation to form a continuous production line.

Thereafter, the raw material filled tray is conveyed by the conveyor through the disinfection station 2 wherein a plant essential oil (PEO) emulsion, such as a phenol emulsion, is added to the substrate of raw material in an amount sufficient to effectively inhibit microbial growth on the substrate, i.e. to disinfect the substrate of microbes, and to form a mixture therewith. The emulsion is characterized in being able not only to inhibit microbial growth on the substrate but also to disinfect the raw material of any microbial growth thereon by inactivating potential contaminates.

The emulsion that is used includes a plant essential oil (PEO) selected from the group consisting of cinnamon, clove, lemongrass, oregano, savory, spearmint, tea tree, and thyme and is provided in volumetric concentrations of 0.1 to 2.0% in water. The ratio of phenol emulsion to substrate may be in the range of 500 ml to 1000 ml of phenol emulsion to 50 g to 500 g (grams) of substrate. The greater the volumetric concentration of the PEO in the emulsion, the lower the amount of emulsion relative to the substrate.

After adding of the phenol emulsion to the substrate in the tray, a lid is placed over the open top of the tray to maintain the contents of the tray in a sealed condition and the sealed tray is held for a time sufficient for the PEO to disinfect the raw material of any microbial growth thereon by inactivating potential contaminates. For example, the time may vary from 15 minutes to 180 minutes.

Alternatively, the raw materials can be disinfected in the hopper by means of a batch process that is agitated to maintain the plant essential oil emulsion and then conveyed to an inoculation stage.

The raw materials can also be continuously disinfected during the conveyance stage, in which the plant essential oil emulsion is recirculated in an enclosed auger conveyor to immerse the raw materials while the materials are transported.

The substrate is held in the batch or continuous immersion process for a sufficient period to inhibit previously existing microbes, between 15 minutes and 180 minutes. The emulsion can be reused in either the batch or continuous process by reclaiming the fluid once substrate is removed.

Upon completion of the disinfection stage, the mixture of substrate and PEO is delivered to the inoculation station 3 in which an inoculum including a preselected fungus is added to disinfected mixture and the inoculated fungus is allowed to grow hyphae. At this time, the lid of the tray is removed to allow the inoculum to be inoculated into the substrate, for example, by spraying of the inoculum over the top surface of the substrate and thereafter the lid is placed back on the tray to seal the inoculated substrate.

Alternatively, the inoculum can be applied to the mixture of substrate and PEO prior to placement of the lid on the tray in the disinfection stage, that is, the substrate may be placed said in a sealed condition after the emulsion and inoculum are added to substrate.

The inoculum includes a fungus such as a fungus selected from the white rot decomposers that produce the desired enzyme. The families are Polyporaceae and Ganodermataceae, while the order is Aphyllophorales. For example, the preferred fungus is selected from the group consisting of the two species, Ganoderma resinaceum and Fomes fomentarius which are known as white rot decomposers and metabolize the aromatic biopolymer lignin and the two species, Ganoderma lucidum and Ganoderma brownii which are known for producing peroxidases and/or dehydrogenases for metabolizing phenols and aldehydes, respectively.

The inoculum is in liquid form with a concentration of 1% to 75% fungus to growth medium. The inoculum can also be grain spawn, in which the fungal tissue is carried on a solid media, such as winter rye grain or millet.

The amount of inoculum added to the substrate mixture is 10% to 32% by volume of the inoculated substrate depending on whether a liquid or solid tissue application is used

After inoculation of the substrate with the inoculum, the sealed tray is delivered to the incubation station 4 in which the fungus is allowed to grow hyphae and the hyphae allowed to form a network of interconnected mycelia cells through and around the discrete particles of the substrate thereby bonding the discrete particles together to form a self-supporting sterilized composite material.

Depending on the materials involved, the incubation time may vary from five (5) days to fourteen (14) days.

The emulsion can also be reused a number of times (upwards of three reuses) without loss of efficacy of the emulsion's disinfection characteristics. The reuse is defined by two current methods: a batch process in which disinfected substrate is removed and new substrate is applied; or a continuous process in which the substrate flows through a conveyance system (auger) with a dwell time of, for example, 30 minutes and the emulsion is re-circulated with new oil and water applied to account for consumed compounds.

For example, referring to FIG. 2 wherein like reference characters indicate like parts as above, the PEO emulsion that is not absorbed into the raw material may be removed from the disinfection and sealing station 2 after a suitable dwell time for recycling purposes prior to the substrate being delivered to the inoculation station 3.

Alternatively, referring to FIG. 3 wherein like reference characters indicate like parts as above, the remaining PEO emulsion may be recycled to the disinfection station to disinfect newly added substrate. In addition, once the substrate is disinfected, in either a batch process (agitated bath) or in a continuous conveyance system, the substrate can be post processed to devolitize the remaining aromatics in a devolitilization station 2′; although this stage is not entirely necessary. The devolitization process can include, but is not limited to, a water or hydrogen peroxide rinse, a soak in vegetable oil, or a spray of lime solution. As illustrated, the devolitilation step includes removing the lid on the tray containing the substrate, adding a devolitilization agent, e.g. a hydrogen peroxide solution, in order to de-volatize the remaining phenols and aldehydes as well as any water, calcium oxide (lime), or vegetable oil contained in the substrate and then replacing the lid on the tray prior to delivery of the substrate to the inoculation station 3.

The following set forth various examples of the method of the invention.

### EXAMPLE 1

Eight plant essential oils (cinnamon, clove, lemongrass, oregano, savory, spearmint, tea tree and thyme) were prepared with tap water in 600 ml Pyrex glass beakers in the following volumetric concentrations: 0.1, 0.5, 1.0, 1.5, 2.0% [v:v]. Twenty-four grams of a propriety cotton burr substrate was placed in a 25×25 cm square of tule mesh tied with a rubber band to make one bundle. Each concentration was tested in replicates of 5, by submersing each bundle in a beaker containing one of the plant essential (PEO) concentrations for three hours. Each beaker was placed under constant magnetic stirring at 300 rpm, to maintain the oil emulsion during the exposure period.

The bundle was then opened within a laminar flow hood and three pieces of cotton burrs were transferred to five Petri plates each of Potato Dextrose Agar (PDA) and Tryptic Soy Agar (TSA).

The submersion process was repeated for each PEO at the five concentrations over a period of three weeks. The number of ascomycete, zygomycete and bacteria/yeast colonies were recorded for each plate every 3 days for 15 days. The mean number of each contaminant type over the 15 day colonization period among the eight PEOs were compared using a one-way analysis of variance (ANOVA). For each contaminant type there were significant differences among the eight oil/concentration combinations (p<0.0001). Potato dextrose agar selects for growth of ascomycetes and zygomycetes and tryptic soy agar selects for growth of bacteria and yeasts. While all contaminants were observed and recorded for each of the two media, results showed that more ascomycetes and zygomycetes grew on PDA and more pronounced bacterial and yeast growth occurred on TSA.

The two oils most effective at inhibiting zygomycetes were thyme and oregano at 0.5,1.0, 1.5, and 2.0% (p<0.0001, FIG. 2). The sterile control showed no zygomycete contamination while the unsterilized/untreated raw substrate control showed one zygomycete colony on average.

Full inhibition of Ascomycetes occurred on TSA with all five concentrations of cinnamon, lemongrass, oregano, and thyme oils. Savory and tea tree oil inhibited 100% of ascomycete growth on TSA at 0.5, 1.5 and 2.0%. Fewer oils were effective against ascomycetes on PDA; therefore this agar was used to determine minimum inhibitory concentrations (MIC). Cinnamon and lemongrass oils offered full inhibition of ascomycete colonies at 0.5 to 2.0% concentrations (p<0.0001). Oregano oil was effective at 0.5, 1.5 and 2.0% while thyme was effective at 1.0, 1.5 and 2.0% (p<0.0001).

Complete inhibition of bacteria and yeast did not occur on TSA plates with any of the eight PEOs. The sterile control plates averaged 0.6+/−0.6 colonies. A significant reduction in the number of bacterial and yeast colonies compared to the control occurred with 1.0, 1.5 and 2.0% cinnamon oil on PDA (p<0.0001) and with 1.5 and 2.0% cinnamon oil and 1.5% oregano oil on TSA (p<0.000).

In conclusion,
Thyme and oregano oils had equivalent zygomycete inhibition to the sterilized controls at 0.5, 1.0, 1.5, and 2.0% [v:v]. The active phenols in the aforementioned oils are carvacrol and thymol.
Cinnamon and lemongrass oils fully inhibited ascomycete colonies at 0.5 to 2.0% [v:v] concentrations, which contain the active compounds of cinnamaldehyde and limonene respectively. Oregano and thyme oils were equally effective at 1.5 and 2.0%.
Bacteria and yeast were found on both agar media types following the PEO disinfection and sterilization. The oil concentrations that offered comparable or superior disinfection to sterilization were 1.0, 1.5 and 2.0% cinnamon oil on PDA, and with 1.5 and 2.0% cinnamon oil and 1.5% oregano oil on TSA.

In sum, testing determined that Thyme at 0.5%, Oregano at 0.5 and 1.0%, Lemongrass at 1.0%, and Cinnamon at 1.5% were the most effective in inhibiting zygomycetes, ascomycetes and bacteria/yeast at their respective concentrations.

### EXAMPLE 2

Example 2 followed the same procedure as Example 1, but examined the efficacy of the five selected oil concentrations at the following exposure intervals: 30, 60, and 120 minutes. This analysis determined the minimum exposure time required for microbial inhibition on cotton burr substrate.

An additional 180 minute interval was conducted for each concentration followed by two reuse intervals of an equivalent exposure time. This analysis determined the loss of potency of the PEOs with prolonged contamination exposure. The reuse intervals entailed the immersion of three successive substrate bundles in the same oil emulsion.

The disinfected substrate was once again placed on PDA and TSA, and observed for active contamination. A second complete set of each PEO concentration was also treated with 2.5 mL of Tween 80; a surfactant that was found to enhance substrate coverage. The substrate was placed on agar media and observed for microbial growth using the same procedure as the initial study. A Fisher's Exact Test was used to compare the frequency of contamination between substrate treated and untreated with Tween 80. The mean number of colonies over the 15 day period was calculated for each replicate and used in a one way analysis of variance to test for significance between the number of zygomycete, ascomycete or bacteria/yeast contaminants and the five plant oil concentration/time combinations.

There was no significant difference between the frequency of contamination on substrate treated with the surfactant Tween 80 and non-treated substrate in the inhibition of zygomycetes, ascomycetes or bacteria/yeast (p=0.318, 0.499, and 0.448 respectively).

Each of the ANOVAs for zygomycete, ascomycete and bacteria/yeast colonies growth showed significant differences among treatments (p<0.0001) when comparing the various PEO concentrations and submersion time intervals. Thyme oil at 0.5% [v:v] for 30 minutes of submersion was the most effective at inhibiting zygomycetes and ascomycetes. Oregano oil at 1.0% [v:v] was offered full inhibition of all molds at 60 minutes submersion. Cinnamon oil at 1.5% [v:v] deterred 100% of molds at 30 minutes of exposure.

During the reuse trials only the Cinnamon oil at 1.5% and the Oregano oil at 1% [v:v] fully inhibited all mold, ascomycetes and zygomycetes, across all three intervals. The Oregano oil at the 0.5% concentration effectively inhibited all zygomycete growth for all three intervals, while the 1% Lemongrass and 0.5% Thyme [v:v] were only effective against this phyla during the first two intervals. Oregano 0.5%, Lemongrass 1%, and Thyme 0.5% [v:v] can be used twice to inactive ascomycetes prior to losing potency. Only the first use of the 1.5% Cinnamon oil was effective against yeast and bacteria, all other time intervals and concentrations exhibited contamination.

In sum,
The Thyme and Cinnamon oils at 0.5% and 1.5% [v:v], respectively, fully inhibited mold growth within a 30 minute immersion period. This rate of disinfection is comparable to the current pasteurization system used in production.
The Oregano oil was only effective at exposure times comparable to, or in excess of, 60 minutes.
None of the oils were effective at exterminating bacteria or yeast at exposure times below 120 minutes. This contamination rate is comparable to the current pasteurization system, which is not sterilization, and does not have a detrimental effect on basidiomycete mycelium growth.
The 1.5% Cinnamon oil and the 1% Oregano oil concentrations can be reused at least three times without loss of efficacy against lower level fungi. The other three concentrations (Oregano 0.5%, Lemongrass 1%, and Thyme 0.5% [v:v]) can be reused twice to inactivate mold.

The only oil to inhibit 100% of bacterial or yeast colonies on TSA was cinnamon oil at 1.5% over 120, 180 and 360 minutes of substrate submersion.

### EXAMPLE 3

A combination of 0.5% thyme or oregano oil (containing the respective phenols of thymol and carvacrol) and 1.5% cinnamon oil (containing the phenol eugenol and the aldehyde cinnamaldehyde) was tested in an additional experiment.

For the preliminary PEO blend experiment, use was made of the combination of 0.5% thyme oil and various concentrations of cinnamon (0.75%, 0.875%, and 1.0%) or lemongrass oils (1.25% and 1.5%). On PDA, which selects for growth of zygomycetes and ascomycetes, there was a significant difference in the inhibition with 0.875% cinnamon oil inhibiting all growth (p=0.0002). On the TSA plates, the cinnamon oil at 0.875% had the lowest mean number of colonies (1.0), but this number did not significantly differ from the other PEO concentrations or the control (1.4) (p=0.812).

The experiment was repeated using 0.5% oregano oil with one of five concentrations of cinnamon oil added (0.5%, 0.75%, 0.875%, 1.0%, 1.5%). Cinnamon at 1.5% completely inhibited the growth of bacteria and yeast colonies on both PDA and TSA plates compared to the control plates that averaged 1.8 and 1.4 colonies per plate respectively. These results are quite appealing as the PEO blend reaches a level of inhibition not presently achieved with a steam process.

### EXAMPLE 4

This example focused on scaling the PEO disinfection process in order to inoculate the substrate with the mycelium culture. Mycelium growth, which serves as an adhesive and yields the final cohesive product, is a critical metric for determining the applicability of PEO disinfection to the overall process.

Batch processing was conserved from the lab bench scale, and a 5-gallon, watertight, rotary tumbler was instituted to maintain the oil emulsion in the large batch.

Each of the five oil concentrations examined under Example 2 was repeated at the larger scale; applying 1.5 gallons of substrate to the tumbler barrel and 3 liters (L) of the PEO emulsion.

The barrel was rotated to maintain the emulsion for 30 minutes, the equipment was operated in ambient conditions, and the barrel was only opened in a laminar flow hood to prevent background contamination.

Upon completion of the disinfection stage, the substrate was rinsed with one of three post treatments in order to de-volatize the remaining phenols and aldehydes; water, calcium oxide (lime), or vegetable oil. Each of the de-volatization fluids were plated on Potato Dextrose Agar to ensure a microbial contaminate was not carried by the bath, and mold was not observed on these agar plates.

The disinfected substrate was then inoculated with grain spawn carrying Ganoderma resinaceum mycelium at an 8% [v:v] concentration. The substrate volume was sufficient to create nine replicate blocks (8″×5.5″×1″) for each de-volatization fluid. The specimens were tested against a control that was comprised of the same fungal culture and substrate that was sterilized in an autoclave at 15 psi and 121° C. for 1 hour. All specimens were incubated in a class 10,000 laboratory, which offers a constant environment at 25° C. and 20% RH.

Mycelium growth was scored every three days using a 0-5 point system at half-point intervals. A “1” represented 1 cm of radial mycelial growth from the inoculation points; locations of grain spawn. A “2”, “3” and “4” signify ≧25%, 50%, and 75% surface colonization (coverage of growth), respectively. A “5” denotes full colonization of the substrate or completion of primary incubation.

Full colonization by Ganoderma resinaceum was reached in 10 days for the control that was not treated with oil or devolatilised with 75% of 12 replicates contaminated with ascomycetes. The only treatment to equal this 10-day growth time was 0.5% oregano with vegetable oil, however 89% of the nine replicates were contaminated with zygomycetes and ascomycetes. The most promising in regards to disinfection were the Thyme and Oregano oils that were devolatized with lime solution.

The plant essential oil may also be applied to a finished mycological composite to promote anti-microbial properties. For example, the oil emulsion can be applied by fully immersing the mycological composite in an agitated broth or via a sputter coat to cover all surfaces.

More particularly, using the oil emulsion to disinfect completed materials could include: (1) production of the mycological composite, (2) fabrication of an oil emulsion, 2% cinnamon oil [v:v], (3) immersion of the composite in the oil emulsion for 120 minutes, and (4) drying the composite to obtain a less than 10% moisture content measured via electrical conductivity.

Of importance, a comparable colonization rate is not the sole objective of the process of the invention but rather achieving higher product yields is an objective. In relation to the colonized substrate, the thyme and oregano, which colonized the substrate in 14 and 12 days respectively, had far higher yield rates than the control (sterilized). The thyme was 0% failure, and the oregano was 11%, while the control was 75%.

The invention thus provides a simple and economical technique for disinfecting bulk materials that can later be inoculated with a filamentous fungus for composite materials production or cultivation of food (mushrooms).

What is claimed is:

1. A method for producing a composite material comprising the steps of
providing a substrate of a raw material composed of discrete particles of an agricultural byproduct;
adding a plant essential oil emulsion to said substrate in an amount and for a time sufficient to effectively inhibit microbial growth on said substrate and to form a mixture therewith;
thereafter adding an inoculum including a preselected fungus to said mixture; and
allowing the fungus to grow hyphae and to allow the hyphae to form a network of interconnected mycelia cells through and around the discrete particles thereby bonding the discrete particles together to form a self-supporting composite material.

2. A method as set forth in claim 1 wherein said raw material is a ligninocellulosic material.

3. A method as set forth in claim 1 wherein said raw material is a cellulosic waste.

4. A method as set forth in claim 3 wherein said raw material is cotton gin waste.

5. A method as set forth in claim 1 wherein said emulsion is at least one of a phenol emulsion and an aldehyde emulsion,

6. A method as set forth in claim 1 wherein at least a portion of said emulsion is removed from said mixture of raw material and emulsion prior to inoculation with said fungus.

7. A method as set forth in claim 1, wherein said preselected fungus is a white rot decomposer selected from the families Polyporaceae and Ganodermataceae and the order of Aphyllophorales.

8. A method as set forth in claim 7 wherein said preselected fungus is a white rot basidiomycetes.

9. A method as set forth in claim 7-wherein said preselected fungus is selected from the group consisting of Ganoderma resinaceum, Fomes fomentarius, Ganoderma lucidum and Ganoderma brownii.

10. A method as set forth in claim 1 further comprising the step of de-volatizing the substrate of any remaining emulsion prior to said step of adding the inoculum.

11. A method for producing a composite material comprising the steps of providing a substrate of a raw material composed of discrete particles of an agricultural byproduct;
adding a plant essential oil emulsion to said substrate in an amount and for a time sufficient to effectively inhibit microbial growth on said substrate;
thereafter removing the emulsion from said substrate;
thereafter adding an inoculum including a preselected fungus to said substrate; and
allowing the fungus to grow hyphae and to allow the hyphae to form a network of interconnected mycelia cells through and around the discrete particles of said substrate thereby bonding the discrete particles together to form a self-supporting composite material.

12. A method as set forth in claim 11 said substrate is subjected to a devolitization step to remove a residual amount of plant essential oil of said emulsion from said substrate prior to said step of adding said inoculum.

13. A method for producing a composite material comprising the steps of providing a substrate of a raw material composed of discrete particles of an agricultural byproduct;
thereafter adding an inoculum including a preselected fungus to said substrate;
allowing the fungus to grow hyphae and to allow the hyphae to form a network of interconnected mycelia cells through and around the discrete particles of said substrate thereby bonding the discrete particles together to form a self-supporting composite material; and
thereafter applying a plant essential oil emulsion to said self-supporting material in an amount and for a time sufficient to effectively inhibit microbial growth on said self-supporting composite material.

14. In a method for producing a composite material, the steps of
providing a substrate of a raw material composed of discrete particles of an agricultural byproduct;
adding a plant essential oil emulsion to said substrate in an amount and for a time sufficient to effectively inhibit microbial growth on said substrate;
thereafter adding an inoculum including a preselected fungus to said substrate; and
incubating the inoculated substrate for a time sufficient to allow the fungus to grow hyphae to form a network of interconnected mycelia cells through and around the discrete particles.

15. In the method of claim 14 the' further step of removing said emulsion from said substrate prior to said step of adding said inoculum to said substrate.

16. In the method of claim 15 wherein the further step of removing a residual amount of plant essential oil of said emulsion from said substrate by devolitization.

17. In the method of claim 13 the further step of placing said substrate in a sealed condition after said emulsion and said inoculum are added to said substrate and prior to said step of incubating the inoculated substrate.
